(19) United States
(12) Patent Application Publication
(10) Pub. No.: US 2017/0305831 A1
(43) Pub. Date: Oct. 26, 2017

(54) PREPARATION OF A SORBATE ESTER

(71) Applicants: Dow Global Technologies LLC, Midland, MI (US); Rohm and Haas Company, Philadelphia, PA (US)

(72) Inventors: Selvanathan Arumugam, Blue Bell, PA (US); John Ell, Quakertown, PA (US); Bo Lu, Shanghai (CN); Brandon Rowe, Robbinsville, NJ (US); Kenneth E. Stockman, Midland, MI (US); Jiguang Zhang, Shanghai (CN)

(21) Appl. No.: 15/521,319

(22) PCT Filed: Oct. 22, 2014
(86) PCT No.: PCT/CN2014/089162

§ 371 Date: Apr. 24, 2017

(51) Int. Cl. C07C 67/26; C07C 69/587

(52) U.S. Cl. CPC C07C 67/26; C07C 69/587

(57) ABSTRACT

The present invention is a process for preparing a hydroxyalkyl sorbate comprising the steps of of: a) contacting together in a reaction vessel a solvent, sorbic acid, a transition metal halide catalyst, an anti-oxidant, and an alkylene oxide under conditions sufficient to form the hydroxyalkyl sorbate; b) removing the solvent in vacuo, wherein the anti-oxidant is characterized by the following formula or a carboxylic acid salt thereof:

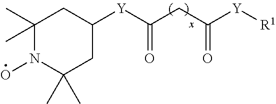

wherein Y, x, and R¹ are defined herein. The process of the present invention provides a convenient way of preparing hydroxyalkyl sorbates in high yield and purity without complicated workup steps.

### BACKGROUND OF THE INVENTION

The present invention relates to the preparation of a sorbate ester, more particularly to the preparation of a hydroxyalkyl sorbate, which is useful as a reactive coalescent in coatings formulations.

Sorbic esters have recently been shown to be suitable as reactive coalescents that promote significant improvement in the coating hardness and tack in waterborne architectural coating formulations. A sorbic ester of particular interest is hydroxypropyl sorbate (sorbic PO), which can be prepared by the FeCl₃ catalyzed reaction of sorbic acid and propylene oxide, as disclosed by Masahiro et al. in EP0387654A2. Masahiro teaches that direct purification of sorbic PO by distillation is problematic because “the heat transfer surface of a distillation apparatus is contaminated by catalyst and the long term operation becomes impossible.” Consequently, multiple washing steps are required prior to distillation. Accordingly, it would be an advance in the art to find a more efficient and cost effective way of preparing hydroxypropyl sorbates such as sorbic PO.

### SUMMARY OF THE INVENTION

The present invention addresses a need in the art by providing a process for preparing hydroxyalkyl sorbate comprising the steps of: a) contacting together in a reaction vessel an organic solvent, sorbic acid, a transition metal halide catalyst, an anti-oxidant, and an alkylene oxide which is a C₂-C₄ alkylene oxide or glycidol under conditions sufficient to form the hydroxyalkyl sorbate; b) removing the solvent in vacuo, wherein the anti-oxidant is characterized by the following formula or a carboxylic acid salt thereof:

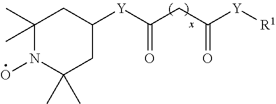

wherein each Y is independently NH or O; x is 0 to 10; and R¹ is H or

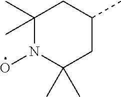

where the dotted line represents the point of attachment to Y.

Hydroxyalkyl sorbates can be prepared in an efficient and cost-effective manner by the process of the present invention.

### DETAILED DESCRIPTION OF THE INVENTION

The present invention is a process for preparing a hydroxyalkyl sorbate comprising the steps of of: a) contacting together in a reaction vessel an organic solvent, sorbic acid, a transition metal halide catalyst, an anti-oxidant, and, and an alkylene oxide which is a C₂-C₄ alkylene oxide or glycidol under conditions sufficient to form the hydroxyalkyl sorbate; b) removing the solvent in vacuo, wherein the anti-oxidant is characterized by the following formula or a carboxylic acid salt thereof:

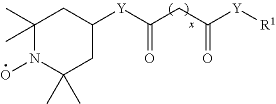

wherein each Y is independently NH or O; x is 0 to 10; and R¹ is H or

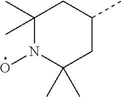

where the dotted line represents the point of attachment to Y.

As used herein, a hydroxyalkyl sorbate refers to hydroxyethyl sorbate, hydroxypropyl sorbate, hydroxybutyl sorbate, or 1,2-dihydroxyethyl sorbate, with hydroxypropyl sorbate being preferred. As used herein, hydroxypropyl sorbate is either 2-hydroxypropyl sorbate or 2-hydroxy-1-methylethyl sorbate, or a combination thereof. The C₂-C₄ alkylene oxides are ethylene oxide, propylene oxide, and butylene oxide, with propylene oxide being preferred.

The solvent is preferably a nonpolar solvent, examples of which include ethyl acetate, butyl acetate, xylenes, toluene, and mesitylene. Examples of suitable transition metal halide catalysts include titanates such as TiCl₄, TiBr₄, and alkoxylated titanates; and halogenated ferric catalysts such FeCl₃, and FeBr₃, with FeCl₃ being preferred. The catalyst is used in a sufficient amount to promote the conversion of the sorbic acid and the propylene oxide to the hydroxypropyl sorbate, preferably from 0.1, more preferably from 0.5 weight percent, to preferably 5, more preferably to 2 weight percent, based on the weight of the sorbic acid and the propylene oxide.

The anti-oxidant is preferably a compound of the following formula:

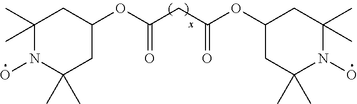

where x is preferably from 2 to 10; more preferably 4 to 8. The anti-oxidant is preferably used in an amount of from 0.01, more preferably from 0.02, more preferably from 0.05 weight percent, to preferably 1, more preferably to 0.5, most preferably to 0.2 weight percent, based on the weight of the sorbic acid. It is understood that when R¹ is H, the anti-oxidant may also be in the form of a carboxylic acid salt.

The solvent, sorbic acid, catalyst, and anti-oxidant are advantageously contacted together in a reaction vessel at an advanced temperature, preferably in a range of from 50° C., more preferably from 65° C., to preferably 140° C., more preferably to 100° C., prior to introduction of the propylene oxide to the reaction vessel. More preferably, the propylene oxide is added slowly to a mixture of the solvent, sorbic acid, catalyst, and anti-oxidant to prevent the formation of oligomeric byproducts and to control the reaction exotherm.

The reaction is preferably carried out to substantial completion, after which time the solvent is removed, preferably in vacuo at an advanced temperature. The product is advantageously purified after solvent removal without any additional workup (for example, by washing) by heating the contents of the flask in vacuo to form a vapor of the desired product at a temperature in the range of from 110° C., preferably from about 150° C. to 220° C., preferably to 200° C., then condensing the vapor in a collection vessel. Because the anti-oxidant has such a high boiling point, the conditions under which the product vaporizes are insufficient to vaporize the anti-oxidant.

The process of the present invention provides for an efficient and cost-effective way of producing high purity hydroxyalkyl sorbates, more particularly hydroxypropyl sorbate, in yields exceeding 90%. In particular, it has been discovered that a purified product can be obtained without time-consuming workup steps. It is believed that the use of the high boiling antioxidant in the process prevents antioxidant carryover in the purification step, which causes gellation in the reaction vessel.

A second anti-oxidant, which may be the same as or different from the anti-oxidant described herein, is advantageously added to the purified product after purification to achieve storage stability. Any suitable anti-oxidant or combinations of anti-oxidants would be effective for this purpose; for example, from 10 ppm to 5000 ppm of hindered N-oxides, preferably TEMPO ((2,2,6,6-tetramethylpiperidin-1-yl)oxidanyl) or 4-hydroxy TEMPO, more preferably 4-hydroxy TEMPO, or hindered phenols such as 2,6-bis(1,1-dimethylethyl)-4-methylphenol are added to the product after purification. More preferably, the addition of a combination of hindered N-oxides and hindered phenols are found to be particularly effective for providing long term storage stability.

### Abbreviation

The anti-oxidant used in the example of the present invention is Prostab 5415 polymerization inhibitor and is characterized by the following structure:

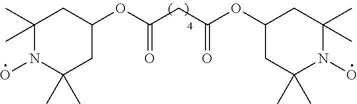

### EXAMPLE

### Preparation of Hydroxypropyl Sorbate

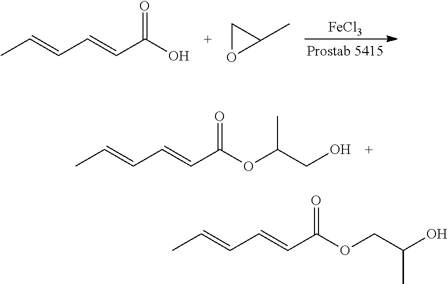

A 500 mL 3-neck flask equipped with a N₂ inlet, a cooling condenser, and a dripping funnel was charged with sorbic acid (92 g, 0.82 mol), xylene (used as a mixture of p-, o-, and m-xylenes, 250 g), FeCl₃ (1.3 g, 0.008 mol) and Prostab 5415 polymerization inhibitor (0.09 g). The vessel was purged with N₂ and the mixture was heated to 85° C. with stirring. Liquid propylene oxide (54 g, 0.93 mol) was added to the mixture at a rate of 1 mL/min, and addition was completed in about 1.5 h. The contents of the vessel were heated for and additional 2 h, after which time the contents of the flask were cooled to 45° C. Solvent was then removed in vacuo for about 1 h. Then the flask and its contents were heated gradually to 160° C. under 10 mm Hg vacuum and the temperature was increased to 180° C., then to 200° C. to bring the liquid to a vapor state and condense it in a separate vessel to form a clear 99.9% pure mixture of 2-hydroxypropyl sorbate and 2-hydroxy-1-methylethyl sorbate (130 g, 93.1% yield).

1. A process for preparing a hydroxyalkyl sorbate comprising the steps of a) contacting together in a reaction vessel an organic solvent, sorbic acid, a transition metal halide catalyst, an anti-oxidant, and an alkylene oxide which is a C₂-C₄ alkylene oxide or glycidol under reaction conditions sufficient to form the hydroxypropyl sorbate; then b) removing the solvent in vacuo;
wherein the anti-oxidant is characterized by the following formula or a carboxylic acid salt thereof: 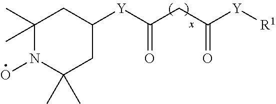
wherein each Y is independently NH or O; x is 0 to 10; and R¹ is H or 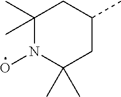
where the dotted line represents the point of attachment to Y.

2. The process of claim 1 wherein the alkylene oxide is propylene oxide.

3. The process of claim 2 wherein the solvent, sorbic acid, and the catalyst are contacted together prior to the introduction of the propylene oxide.

4. The process of claim 3 wherein the process is carried out at a temperature in the range of 50° C. to 140° C., and the transition metal halide is FeCl₃.

5. The process of claim 4 wherein after the solvent removal step, the hydroxypropyl sorbate is purified without additional workup by heating the contents of the flask in vacuo to form a vapor at a temperature in the range of from 110° C. to 220° C., then condensing the vapor in a collection vessel.

6. The process of claim 5 wherein the antioxidant is characterized by the following formula: 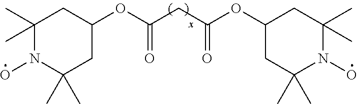
where x is from 2 to 10, and wherein the process is carried out at a temperature in the range of 65° C. to 100° C.

7. The process of claim 6 where x is 8, wherein the anti-oxidant is present at an amount in the range of 0.01 to 0.5 weight percent, based on the weight of the sorbic acid, and wherein the catalyst is present in an amount in the range of 0.1 to 2 weight percent, based on the weight of the sorbic acid and the propylene oxide.

8. A process for preparing a hydroxypropyl sorbate comprising the steps of a) contacting together in a reaction vessel a solvent, sorbic acid, FeCl₃, and an anti-oxidant; then b) adding propylene oxide to the reaction vessel under reaction conditions sufficient to form the hydroxypropyl sorbate; then c) removing the solvent in vacuo; then d) heating the contents of the flask in vacuo to form a vapor at a temperature in the range of from 110° C. to 220° C. and condensing the vapor in a collection vessel to isolate purified hydroxypropyl sorbate; wherein the anti-oxidant is characterized by the following formula: 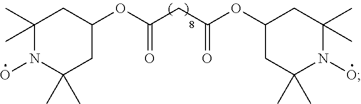
and wherein the anti-oxidant is present at an amount in the range of 0.01 to 0.5 weight percent, based on the weight of the sorbic acid, and wherein the FeCl₃ is present in an amount in the range of 0.1 to 2 weight percent, based on the weight of the sorbic acid and the propylene oxide.

9. The process of claim 8 which further includes the step of adding from 10 ppm to 5000 ppm of a hindered N-oxide or a hindered phenol or a combination thereof.

10. The process of claim 8 which further includes the step of adding from 10 ppm to 5000 ppm of 4-hydroxy TEMPO and 2,6-bis(1,1-dimethylethyl)-4-methylphenol.
